(19) United States
(12) Patent Application Publication
(10) Pub. No.: US 2005/0203124 A1
(43) Pub. Date: Sep. 15, 2005

(54) Compounds for the sustained reduction of body weight

(75) Inventors: Stephane Pollentier, Mainz (DE); Andreas Raschig, Biberach (DE); Juergen Reess, Ulm (DE); Ole Graff, Jyllinge (DK); Birgit Ohrt Mikkelsen, Bindslev (DK); Morten Priskorn, Koebenhavn S. (DK)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignees: Boehringer Ingelheim International GmbH, Ingelheim (DE); NeuroSearch A/S, Ballerup (DK)

(21) Appl. No.: 11/039,991

(22) Filed: Jan. 21, 2005

(30) Foreign Application Priority Data
Jan. 22, 2004 (EP) 04001282
Mar. 11, 2004 (EP) 04005816

(51) Int. Cl. A61K 31/46

(52) U.S. Cl. 514/304

(57) ABSTRACT

The invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof for a medicament for the for the sustained reduction of body weight.

This applications claims priority of EP Application Nos. 04001282 and 04005816, which are incorporated herein by reference in their entirties.

### BACKGROUND OF THE INVENTION

1. Technical Field

The invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof, for the sustained reduction of body weight.

2. Background Information

Excessive food intake generally leads to overweight, i.e., an increase in normal weight which exceeds normal limits. Nowadays, being overweight is not only a serious health risk, but also an economic problem. Overweight is a risk factor for a number of diseases such as high blood pressure, diabetes mellitus, hyperlipidaemia, osteoarthritis, gout, and the associated vascular diseases, particularly arteriosclerosis. Moreover, being overweight can cause emotional problems, including depression.

The only effective therapeutic action is to reduce calorie intake. However, this is difficult to achieve in many patients, in spite of a knowledge of the consequences mentioned above.

Moreover, most of the common treatments for the reduction of body weight achieve a short time reduction, but, in almost all instances, an increase in normal weight that exceeds normal limits is observed soon. Up to now, there has been no effective way to avoid this so-called Yo-Yo effect.

The International patent applications WO 93/09814 and WO 97/30997 disclose tropane derivatives, which are monoamine neurotransmitter re-uptake inhibitors. Moreover, the International patent application WO 97/30997 suggests that such tropane derivatives may also be used to treat obesity. However, there is no indication that a sustained reduction of the body weight could be achieved with the aid of such compounds.

The tropane derivatives for use according to the invention may in particular be tropane derivatives such as those disclosed by patent applications EP 604355, EP 604352, U.S. Pat. No. 5,444,070, EP 604354, WO 95/28401, and WO 97/30997, all of which are encorporated herein in their entirties.

The objective of the invention is to make it easier for the patient to reduce their body weight without suffering from the Yo-Yo effect and thus reduce the health risks associated with overweight.

### BRIEF SUMMARY OF THE INVENTION

It has now been shown, surprisingly, that monoamine neurotransmitter re-uptake inhibitors comprising a 2,3-disubstituted tropane moiety can be used for the sustained reduction of body weight.

Accordingly, the present invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof for a medicament for the sustained reduction of body weight.

### BRIEF DESCRIPTION OF THE DRAWINGS

FIG. 1 is a table showing the change is weight, in kilograms, induced by different doses of a monoamine neurotransmitter re-uptake inhibitor according to the present invention.

FIG. 2 is a table showing the change is weight, as a percentage, induced by different doses of a monoamine neurotransmitter re-uptake inhibitor according to the present invention.

FIG. 3 is a graph showing the absolute change in weight, in kilograms, induced in patients by different doses of a monoamine neurotransmitter re-uptake inhibitor 28 days after the beginning of the treatment.

FIG. 4 in a graph showing the absolute change in weight, in kilograms, induced in patients by different doses of a monoamine neurotransmitter re-uptake inhibitor 56 to 70 days after the begin of the treatment.

FIG. 5 is a graph showing the relative change in weight, as a percentage, induced in patients by different doses of a monoamine neurotransmitter re-uptake inhibitor 28 days after the beginning of the treatment.

FIG. 6 is a graph showing the relative change in weight, as a percentage, induced in patients by different doses of a monoamine neurotransmitter re-uptake inhibitor 56 to 70 days after the beginning of the treatment.

### DETAILED DESCRIPTION OF THE INVENTION

As a rule the monoamine neurotransmitter re-uptake inhibitor comprising a 2,3-disubstituted tropane moiety are those which are disclosed by International patent applications WO 93/09814 and WO 97/30997.

For use according to the invention, it is preferable to use a compound of the general formula (I) 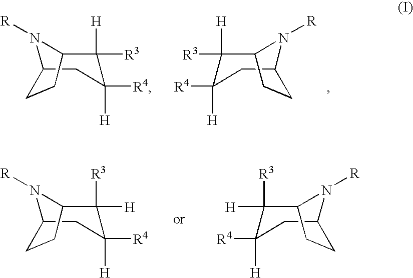
or a pharmaceutical acceptable addition salt thereof, or the N-oxide thereof, wherein
R is hydrogen, alkyl, alkenyl, alkynyl, cycloalkyl, cycloalkylalkyl or 2-hydroxyethyl;
R³ is
   CH₂—X—R′, wherein
      X is O, S, or NR″; wherein
         R″ is hydrogen or alkyl; and
      R′ is
         alkyl, alkenyl, alkynyl, cycloalkyl, cycloalkylalkyl, or-CO-alkyl;
         heteroaryl, which may be substituted one or more times with alkyl, cycloalkyl, or cycloalkylalkyl;
         phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl, which may be substituted one or more times with substituents selected from the group consisting of halogen, CF₃, CN, alkoxy, alkyl, alkenyl, alkynyl, amino, nitro, and heteroaryl; or benzyl, which may be substituted one or more times with substituents selected from the group consisting of halogen, CF₃, CN, alkoxy, alkyl, alkenyl, alkynyl, amino, nitro, and heteroaryl; or
   (CH₂)ₙCO₂R¹¹, COR¹¹, or CH₂R¹², wherein
      R¹¹ is
         alkyl, cycloalkyl, or cycloalkylalkyl;
         phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl or O-thienyl, which may be substituted one or more times with substituents selected from the group consisting of halogen, CF₃, CN, alkoxy, alkyl, alkenyl, alkynyl, amino, nitro, and heteroaryl; or benzyl;
      n is 0 or 1; and
      R¹² is
         O-phenyl, which may be substituted one or more times with substituents selected from the group consisting of halogen, CF₃, CN, alkoxy, alkyl, alkenyl, alkynyl, amino, nitro, and heteroaryl; or
         O—CO-phenyl, which may be substituted one or more times with substituents selected from the group consisting of halogen, CF₃, CN, alkoxy, alkyl, alkenyl, alkynyl, amino, nitro, and heteroaryl; or
   CH═=NOR′, wherein
      R′ is
         hydrogen or O-hydrogen;
         alkyl, O-alkyl, cycloalkyl, cycloalkylalkyl, alkenyl, alkynyl or aryl, all of which may be substituted with-COOH;
         —COO-alkyl;
         —COO-cycloalkyl; or
         phenyl, which may be substituted one or more times with substituents selected from the group consisting of halogen, CF₃, CN, alkyl, cycloalkyl, alkoxy, cycloalkoxy, alkenyl, alkynyl, amino, and nitro;
R⁴ is
   3,4-methylenedioxyphenyl; or
   phenyl, benzyl, naphthyl, or heteroaryl, all of which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

In a special embodiment of the compound of general formula (I), R³ is
1,2,4-oxadiazol-3-yl, which may by substituted in the 5 position with alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl; or
   benzyl, which may be substituted one or more times with substituents selected from the group consisting of halogen, CF₃, CN, alkoxy, alkyl, alkenyl, alkynyl, amino, nitro, and heteroaryl; or
1,2,4-oxadiazol-5-yl, which may by substituted in the 3 position with alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl;
   benzyl, which may be substituted one or more times with substituents selected from the group consisting of halogen, CF₃, CN, alkoxy, alkyl, alkenyl, alkynyl, amino, nitro, and heteroaryl;
   pyridyl, which may be substituted one or more times with substituents selected from the group consisting of halogen, CF₃, CN, alkoxy, alkyl, alkenyl, alkynyl, amino, nitro and heteroaryl; or
   thienyl, which may be substituted one or more times with substituents selected from the group consisting of halogen, CF₃, CN, alkoxy, alkyl, alkenyl,alkynyl, amino, nitro and heteroaryl.

In a further special embodiment of the compound of general formula (I), R³ is
CH₂—X—R′, wherein
   X is
      O, S, or NR″; wherein
         R″ is hydrogen or alkyl; and
   R′ is alkyl, alkenyl, alkynyl, cycloalkyl, cycloalkylalkyl, or-CO-alkyl.

In a still further embodiment of the compound of general formula (I), R³ is
CH═NOR′, wherein
   R′ is
      hydrogen;
      alkyl, cycloalkyl, cycloalkylalkyl, alkenyl, alkynyl or aryl, all of which may be substituted with —COOH;
      —COO-alkyl;
      —COO-cycloalkyl; or
      phenyl, which may be substituted one or more times with substituents selected from the group consisting of halogen, CF₃, CN, alkyl, cycloalkyl, alkoxy, cycloalkoxy, alkenyl, alkynyl, amino, and nitro.

In a further special embodiment of the compound of general formula (I), R⁴ is phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

In a more special embodiment, R⁴ is phenyl substituted once or twice with chlorine.

In a further special embodiment, the tropane derivative having dopamine reuptake inhibitor activity is a (1R,2R,3S)-2,3-disubstituted tropane derivative of formula (I).

In a still further embodiment, the tropane derivative having dopamine reuptake inhibitory activity is a compound of general formula (I), wherein
R³ is
   —CH₂—X—R′, wherein X is O or S, and R′ is methyl, ethyl, propyl, or cyclopropylmethyl;
   —CH═NOR′, wherein R′ is hydrogen or alkyl, or
   1,2,4-oxadiazol-5-yl, which may by substituted in the 3 position with alkyl.

In a still further embodiment, the tropane derivative having dopamine reuptake inhibitory activity is a compound of general formula (I) wherein R is hydrogen, methyl, ethyl or propyl.

In a still further embodiment, the tropane derivative having dopamine reuptake inhibitory activity is a compound of general formula I wherein R⁴ is 3,4-dichlorophenyl.

Preferably those monoamine neurotransmitter re-uptake inhibitor comprising a 2,3-disubstituted tropane moiety are compounds of formula (I1) 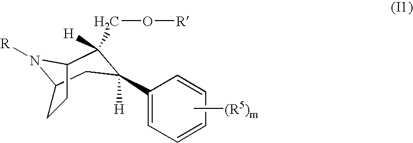
wherein
R represents a hydrogen atom or a C₁₋₆ alkyl group, preferably a hydrogen atom, a methyl or an ethyl group;
R⁵ each independently represents a halogen atom or a CF₃ or cyano group, preferably a fluorine, chlorine or bromine atom;
R represents a hydrogen atom or a C₁₋₆ alkyl or C₃₋₆-cycloalkyl-C₁₋₃-alkyl group, preferably a methyl, ethyl or n-propyl group; and
m is 0 or an integer from 1 to 3, preferably 1 or 2;
   or a tautomer, a pharmaceutically acceptable salt, solvate, or physiologically functional derivative thereof.

As used herein, the expression “C₁₋₆ alkyl” includes methyl and ethyl groups, and straight-chained and branched propyl, butyl, pentyl, and hexyl groups. Particular alkyl groups are methyl, ethyl, n-propyl, isopropyl, and t-butyl.

The expression “C₃₋₆ cycloalkyl,” as used herein, includes cyclic propyl, butyl, pentyl, and hexyl groups, such as cyclopropyl and cyclohexyl.

The term “halogen,” as used herein, includes fluorine, chlorine, bromine, and iodine, of which fluorine and chlorine are preferred.

The term “physiologically functional derivative,” as used herein, includes derivatives obtained from the compound of formula (I) under physiological conditions, these are, for example, N-oxides, which are formed under oxidative conditions.

The term “pharmaceutically acceptable acid addition salt,” as used herein, includes those salts that are selected from among the acid addition salts formed with hydrochloric acid, hydrobromic acid, sulphuric acid, phosphoric acid, methanesulphonic acid, acetic acid, fumaric acid, succinic acid, lactic acid, citric acid, tartaric acid, and maleic acid, the salts obtained from hydrochloric acid, hydrobromic acid, sulphuric acid, phosphoric acid, and acetic acid being particularly preferred. The salts of citric acid are of particular significance.

In a special embodiment, the tropane derivative having dopamine reuptake inhibitor activity is a compound of the general formula (I) selected from:
(1R,2R,3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2R,3S)-3-(3,4-Dichlorophenyl)-tropane-2-O-benzyl-aldoxime;
(1R,2R,3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2R,3S)-3-(3,4-Dichlorophenyl)-tropane-2-O-(1-ethoxycarbonyl-1,1-dimethyl-methyl)-aldoxime;
(1R,2R,3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R,3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoximehydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)-N-Normethyl-2-methoxymethyl-3-(4-chlorophenyl)-tropane;
(1R,2R,3S)-2-Ethoxymethyl-3-(4-chlorophenyl)-tropane;
(1R,2R,3S)-N-Normethyl-2-methoxymethyl-3-(3,4-dichlorophenyl)-tropane;
(1R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2R,3S)-N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2 R,3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R,3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2R,3S)-N-Normethyl-N-allyl-2-(3-(4-pyridyl)-1,2,4-oxadiazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R,3S)-N-Normethyl-2-(3-(4-pyridyl)-1,2,4-oxadiazol-5-yl)-3-(3,4-dichlorophenyl)-tropane;
(1R,2R, 3S)-N-Normethyl-N-allyl-2-(3-(3-pyridyl)-1,2,4-oxadiazol-5-yl)-3-(3,4-dichlorophenyl)-tropane;
(1R,2R,3S)-N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(4-chlorophenyl)-tropane;
(1R,2R,3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R,3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane;
(1R,2R,3S)-2-(4-Fluoro-benzoyl)-3-(4-fluorophenyl)-tropane; or
a pharmaceutically acceptable addition salt thereof.

Most preferred are the compounds of formulas (IA) and (IB)

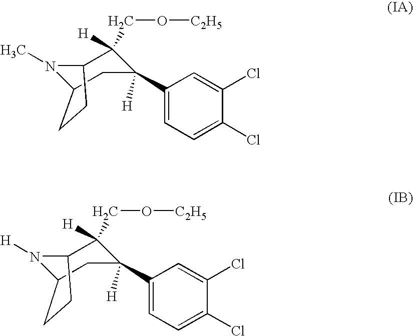

It is particularly preferable to use the monoamine neurotransmitter re-uptake inhibitor comprising a 2,3-disubstituted tropane moiety to prepare a pharmaceutical composition for the reduction of body-weight in cases of slight or heavy overweight.

It is also preferred to use the above mentioned monoamine neurotransmitter re-uptake inhibitor comprising a 2,3-disubstituted tropane moiety to prepare a pharmaceutical composition for the sustained reduction of body weight in healthy persons, as well as in patients with other diseases, such as Pakinson's disease, or in major depressive disorders, or in attention deficit, hyperactivity disorder (ADHD), or in type 2 diabetes patients. Preferably, the patients are male or female adults or elderly people of any race, in particular aged 45 to 95, most preferred aged 60 to 80.

It is particularly preferred to use the above mentioned monoamine neurotransmitter re-uptake inhibitors comprising a 2,3-disubstituted tropane moiety to prepare a pharmaceutical composition for continuous administration for the sustained reduction of body weight.

It is furthermore preferred to use the above mentioned monoamine neurotransmitter re-uptake inhibitor comprising a 2,3-disubstituted tropane moiety to prepare a pharmaceutical composition for transdermal administration for the sustained reduction body weight.

The monoamine neurotransmitter re-uptake inhibitors of formulas (I) and (I1) that are preferably used within the scope of the present invention may optionally be used in the form of their pharmacologically acceptable acid addition salts, and optionally in the form of the hydrates and solvates.

The monoamine neurotransmitter re-uptake inhibitors of formulas IA and IB that may be used according to the invention are preferably used in the form of the pharmaceutically acceptable acid addition salt thereof, and optionally in the form of the hydrates and solvates.

By the pharmaceutically acceptable acid addition salts are meant, according to the invention, those salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, the salts of hydrochloric acid, hydrobromic acid, sulphuric acid, phosphoric acid and acetic acid being particularly preferred. The salts of citric acid are of particular significance.

In the case of the compounds of formulas (IA) and (IB), the use of which is particularly preferred according to the invention, the citrate is of particular importance. For transdermal administration it is preferable to use the base of the compounds of formula (I).

The monoamine neurotransmitter re-uptake inhibitor comprising a 2,3-disubstituted tropane moiety, preferably the compounds of formula (I), most preferably of formulas IA and IB, which may be used according to the invention, may optionally be used in conjunction with other active substances. Preferred combination partners are compounds selected from the categories of the D₁-, D₂-, D₃- or D₄-agonists, anorectics, lipase inhibitors, and sympathomimetics, or a pharmaceutically acceptable salt, solvate, or physiologically functional derivative thereof, selected from among group consisting of adrogolide, A-86929, Rotigotine, NeurVex, nolomirole, pramipexole, talipexol, CHF 1512, (−)-stepholidine, DAR-201, diacrin/Genzyme, bromocriptine, bupropion, LEK-8829, BAM-1110, AIT-203, terguride, aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, ropinirole, SKF-82958, SKF-77434, DU 127090, SLV-308, SLV 318, NeuroCRIB, SP-1037C, spheramine, gallotrank, preclamol, DAB-452, YM-435, BP-897, ProSavin, etilevodopa, P63, A 68930, A 77636, alaptide, alentemol, Cl 1007; PD 143188, BLSI, JA 116a; JA 116, melevodopa; levodopa methyl; CHF 1301; NSC 295453; levomet, MR 708, PD 128483, RD 211, SKF 38393, SKF 81297, U 86170F, U 91356A, WAY 124486, Z 15040, silbutramine, orlistat, amfepramon-HCl, and ephedrine. The dosages of the individual components can be reduced, thanks to the synergistic (additive and magnifying) effects obtained when combinations containing one of the additional active substances in addition to the dopamine receptor agonists according to the invention are used as envisaged.

The novel activity of the monoamine neurotransmitter re-uptake inhibitor comprising a 2,3-disubstituted tropane moiety according to the invention will be illustrated by means of the following Examples using the compound of formula IA including its active metabolite, formula IB. They serve merely to illustrate the invention and are not to be regarded as limiting.

The dosage of said monoamine neurotransmitter re-uptake inhibitor comprising a 2,3-disubstituted tropane moiety according to the invention is naturally highly dependent on the severity of the symptoms to be treated on the one hand and the choice of active substance on the other hand. For example, without restricting the subject matter of the present invention thereto, some possible dosages, especially for the compounds of formulas IA and IB, that are particularly preferred according to the invention will now be given. This may be used in dosages of about 0.05 to 10 mg, preferably about 0.1 to 2.0 mg, in particular about 0.125 to 1.0 mg daily, or 0.1 to 5 mg once weekly. These dosages are based on the compound of formula IA in the form of its free base. Based on the salt form which is preferably used, namely the citrate, the above mentioned dosages correspond to about 0.08 to 16 mg, preferably 0.16 to 2.38 mg, in particular about 0.20 to 1.58 of the compound of formula IA citrate per day.

One possible dosing method, which is described solely as an illustrative example, is described hereinafter (based on the compound of formula IA in the form of its free base) with or without individual dosage titration at weekly intervals depending on the activity and tolerance levels.

The monoamine neurotransmitter re-uptake inhibitors comprising a 2,3-disubstituted tropane moiety may be administered for the purposes according to the invention by oral, transdermal, intrathecal, inhalative, nasal, or parenteral route, preferably by transdermal or parenteral route, most preferably by transdermal route. Suitable preparations include, for example, tablets, particularly slow release tablets, capsules, suppositories, solutions, syrups, emulsions, dispersible powders, implants, or plasters, most preferably micronal plasters. In connection with possible embodiments of a transdermal preparation that may be used according to the invention reference is hereby made. Tablets may be obtained, for example, by mixing the active substance or substances with known excipients, e.g., inert diluents, such as calcium carbonate, calcium phosphate, or lactose, disintegrants, such as maize starch or alginic acid, binders, such as starch or gelatine, lubricants, such as magnesium stearate or talc, and/or agents for obtaining delayed release, such as carboxymethylcellulose, cellulose acetate phthalate, or polyvinylacetate. The tablets may also consist of several layers.

Some examples of pharmaceutical preparations which may be used preferably for formulas IA and IB that may be used according to the invention are given below. These are intended solely as illustrations by way of example without restricting the subject matter of the invention thereto.

Tablet 1:

| Ingredients: | mg |
|---|---|
| Compound of formula IA | 1.00 |
| Mannitol | 121.50 |
| Maize starch | 79.85 |
| Highly dispersed silicon dioxide, anhydrous | 2.30 |
| Polyvidon K25 | 2.35 |
| Magnesium stearate | 3.00 |
| Total | 210.00 |

Tablet 2:

| Ingredients: | mg |
|---|---|
| Compound of formula IA | 0.5 |
| Mannitol | 122.0 |
| Maize starch, dried | 61.8 |
| Maize starch | 18.0 |
| Highly dispersed silicon dioxide, anhydrous | 2.4 |
| Polyvidon K25 | 2.3 |
| Magnesium stearate | 3.0 |
| Total | 210.0 |

Tablet 3:

| Ingredients: | mg |
|---|---|
| Compound of formula IA | 0.25 |
| Mannitol | 61.00 |
| Maize starch | 39.90 |
| Highly dispersed silicon dioxide, anhydrous | 1.20 |
| Polyvidon K25 | 1.15 |
| Magnesium stearate | 1.5 |
| Total | 105.00 |

Tablet 4:

| Ingredients: | mg |
|---|---|
| Compound of formula IA | 0.125 |
| Mannitol | 49.455 |
| Maize starch, dried | 25.010 |
| Maize starch | 7.300 |
| Highly dispersed silicon dioxide, anhydrous | 0.940 |
| Polyvidon K25 | 0.940 |
| Magnesium stearate | 1.230 |
| Total | 85.000 |

Solution for injection:

| Ingredients: | | |
|---|---|---|
| Compound of formula IA | 0.3 | mg |
| sodium chloride | 0.8 | mg |
| benzalkonium chloride | 0.01 | mg |
| water for injections | ad 100 | ml |

Clinical Studies have been carried out in order to compare the maintenance doses of the compounds of formula IA, including its metabolite IB, that were used in 3 studies.

The studies were randomized, double-blind, placebo-controlled per ascending dose group. The studies were designed to assess the safety, tolerability, phamacokinetics (PK), and preliminary efficacy of multiple ascending doses of the compound of formula IA in patients with possible Alzheimer's Disease (AD).

Two studies were performed consecutively with 0.125/0.25 and 0.5/1.0 mg of the compound of formula IA daily every morning to assess the safety and preliminary efficacy of multiple given for 28 days in those patients. After the completion of the first two studies a second study was initiated in elderly volunteers in order to investigate the safety and PK of a median dose of 0.75 mg of the compound of formula IA daily in the morning than were used in the first two studies. Depending on the maintenance dose an at least double loading dose was administrated for up to 6 days.

The objectives of the studies, as stated in the protocols, were:

To determine the safety, tolerability of ascending multiple doses of the compound of formula IA (5 dose levels) given for 28 days, and to determine the preliminary multiple dose (4-week) pharmacokinetics (PK) of the compound of formula IA.

For all three studies, following screening procedures and baseline assessments, eligible patients were admitted to the study center and randomly assigned to receive either compound of formula IA within a total of 5 dose groups or placebo. All patients, including male and female aged 55 to 80 years, participated in an inpatient phase, consisting of acclimatization, loading dosing, and maintenance dosing, prior to the outpatient maintenance dosing phase. The length of the inpatient phase varied depending on the dose group. Following the inpatient phase of the studies, all patients had weekly return visits to the study center on an outpatient basis through Day 28 and then follow-up visits at Days 42 and 56.

A total of 78 patients participated in these studies; 58 patients received compound of formula IA and 20 patients received placebo.

Safety assessments included adverse event profile, physical examination, vital sign measurements (weight, temperature, heart rate, blood pressure (BP) (supine and standing)), clinical laboratory assessments, and others.

All safety analyses were conducted on the safety population, defined as all patients who received at least 1 dose of study medication. Descriptive statistics were provided among other things for vital signs and clinical laboratory assessments.

Descriptive statistics, including number of observations, arithmetic mean, standard deviation, minimum, maximum, arithmetic coefficient of variation, geometric mean (gmean), and geometric coefficient of variation (gCV), were provided for plasma concentrations and pharmacokinetic parameters (only maintenance dose group of 1 mg of the compound of formula IA).

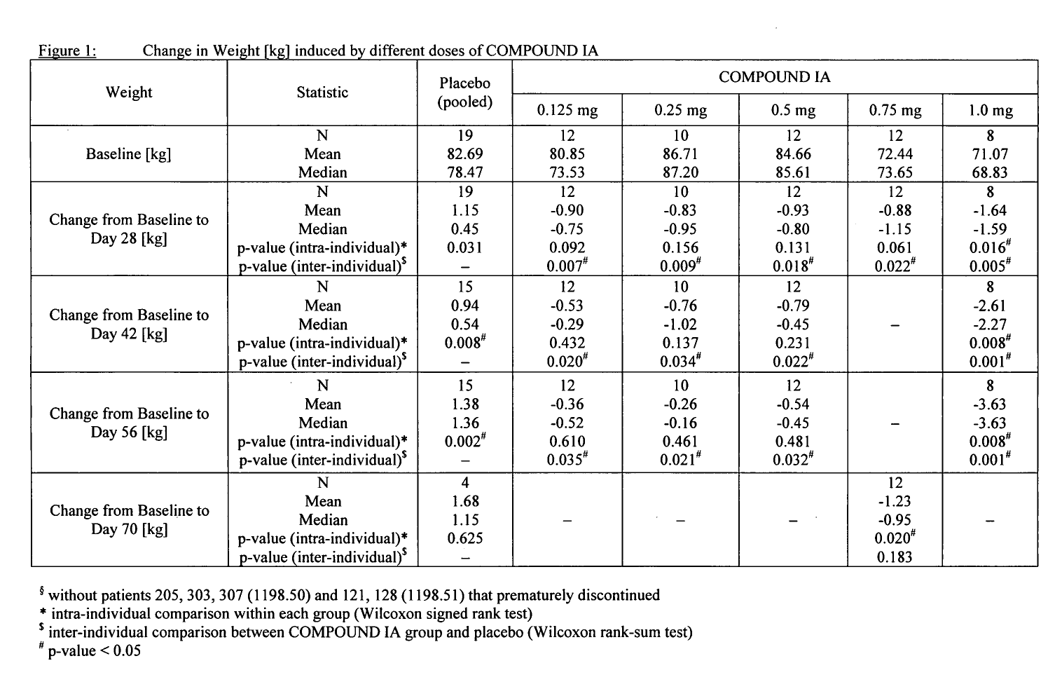

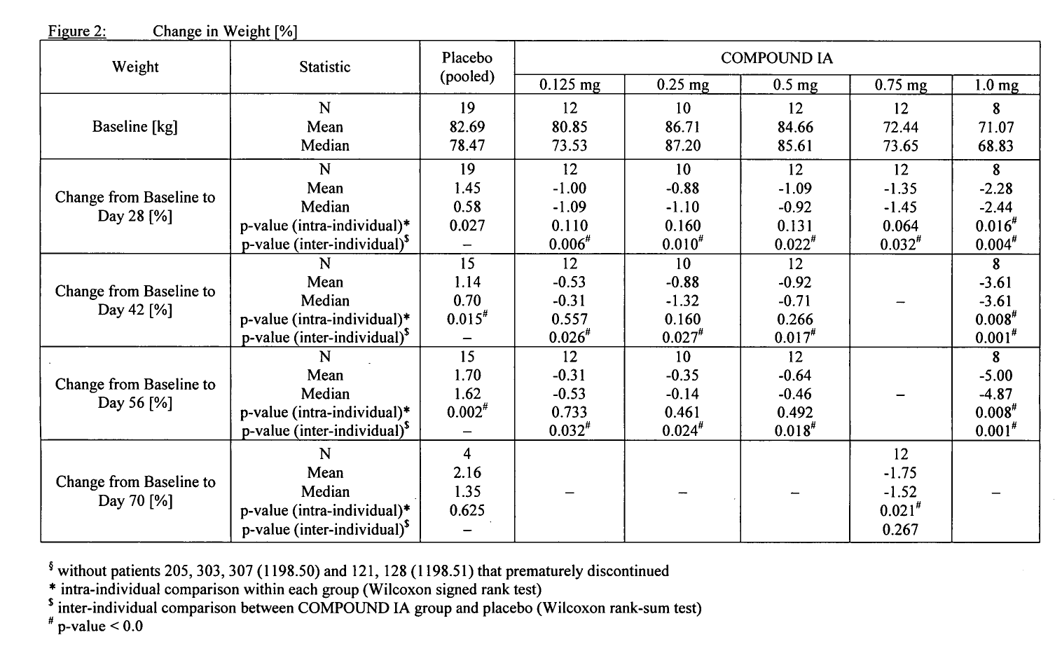

The results regarding the monitoring of the body weight are shown in FIGS. 1 and 2.

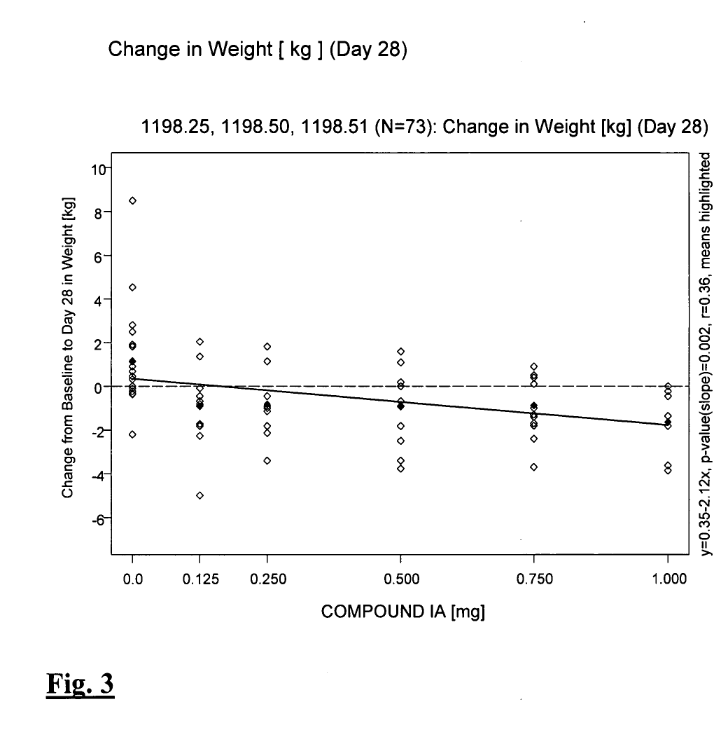

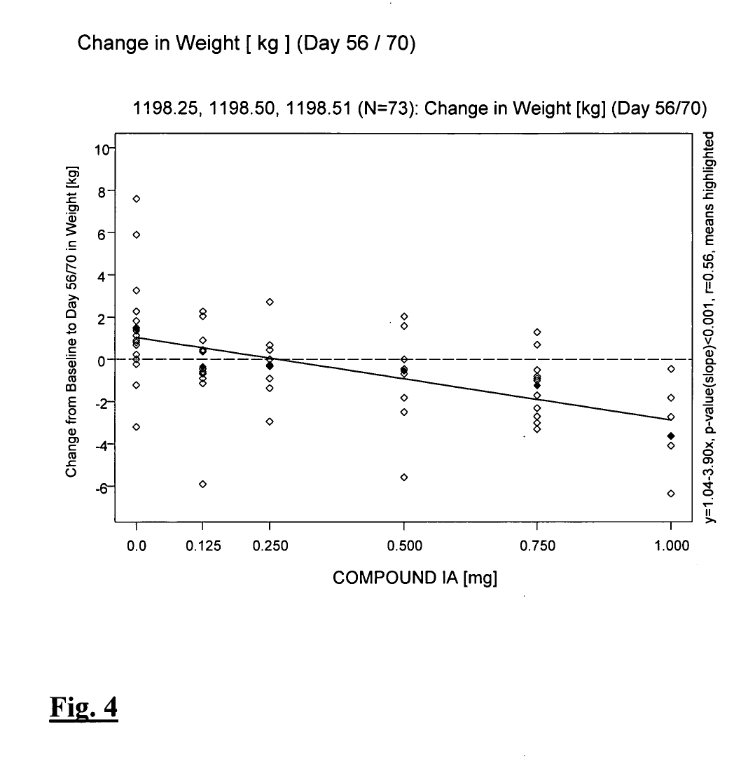

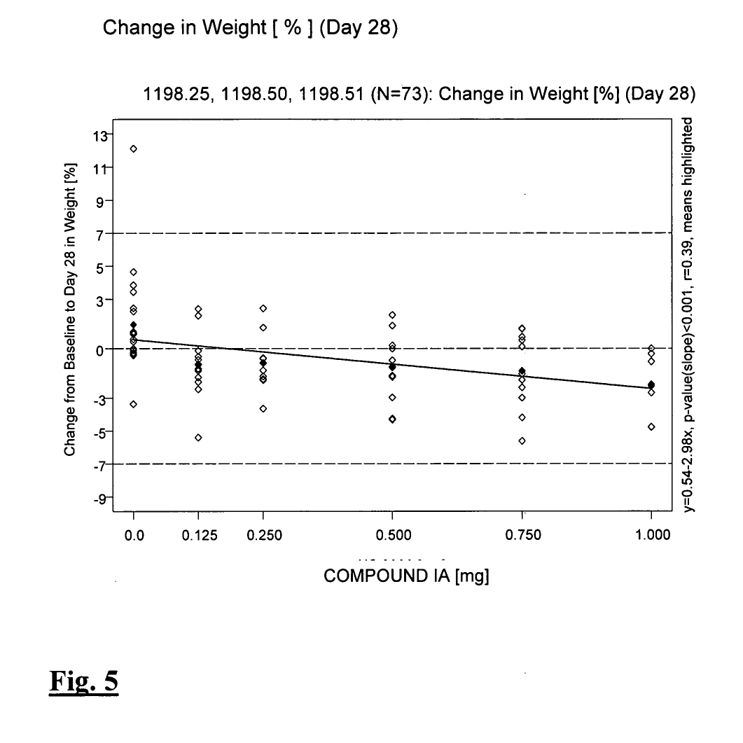

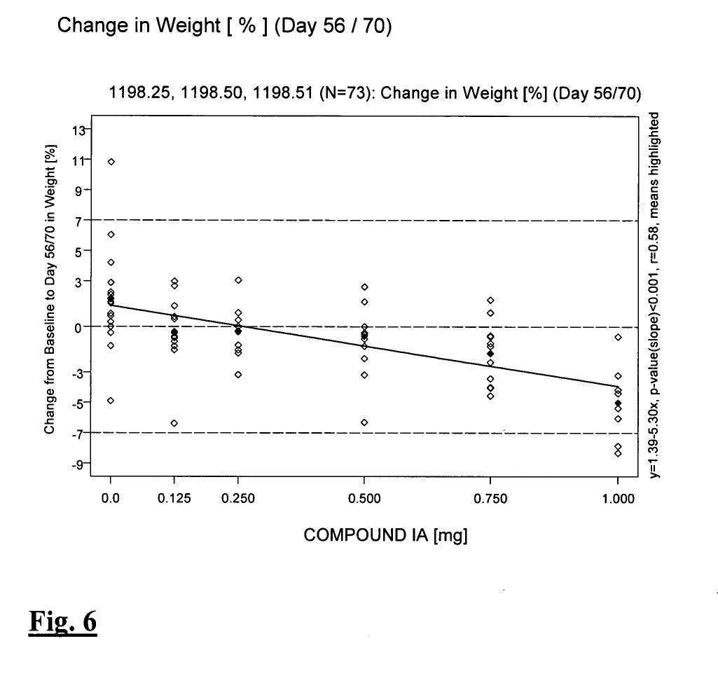

The results are also shown graphically in the FIGS. 3 to 6. From FIGS. 3 and 4, it can be seen that the treatment with the compound of formula IA clearly reduces the bodyweight. Moreover, FIGS. 5 and 6 show that the body weight is still further reduced for several weeks even after the treatment with said compound has been stopped.

1. A method for a sustained reduction of body weight comprising:
administering to a human a composition comprising:
a 2,3-disubstituted tropane moiety, or a tautomer, pharmaceutically acceptable salt, solvate, or physiologically functional derivative thereof.

2. The method according to claim 1, wherein the 2,3-disubstituted tropane moiety is a compound of formula (I) 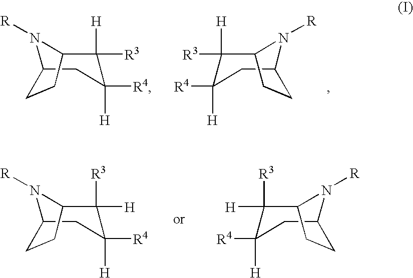
or an addition salt or N-oxide thereof, wherein
R is hydrogen, alkyl, alkenyl, alkynyl, cycloalkyl, cycloalkylalkyl, or
2-hydroxyethyl;
R³ is
CH₂—X—R′, wherein
X is O, S, or NR″, wherein
R″ is hydrogen or alkyl; and
R′ is
alkyl, alkenyl, alkynyl, cycloalkyl, cycloalkylalkyl, or —CO-alkyl;
heteroaryl, which may be substituted one or more times with alkyl, cycloalkyl, or cycloalkylalkyl; phenyl, which may be substituted one or more times with substituents selected from the group consisting of halogen, CF₃, CN, alkoxy, alkyl, alkenyl,alkynyl, amino, nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl, which may be substituted one or more times with substituents selected from the group consisting of halogen, CF₃, CN, alkoxy, alkyl, alkenyl, alkynyl, amino, nitro, and heteroaryl; or
benzyl, which may be substituted one or more times with substituents selected from the group consisting of halogen, CF₃, CN, alkoxy, alkyl, alkenyl, alkynyl, amino, nitro, and heteroaryl; or
(CH₂)ₙCO₂R¹¹, COR¹¹, or CH₂R¹² wherein
R¹¹ is
alkyl, cycloalkyl, or cycloalkylalkyl;
phenyl, which may be substituted one or more times with substituents selected from the group consisting of halogen, CF₃, CN, alkoxy, alkyl, alkenyl, alkynyl, amino, nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl or O-thienyl, which may be substituted one or more times with substituents selected from the group consisting of halogen, CF₃, CN, alkoxy, alkyl, alkenyl, alkynyl, amino, nitro, and heteroaryl; or benzyl;
n is 0 or 1; and
R¹² is
O-phenyl, which may be substituted one or more times with substituents selected from the group consisting of halogen, CF₃, CN, alkoxy, alkyl, alkenyl, alkynyl, amino, nitro, and heteroaryl; or
O—CO-phenyl, which may be substituted one or more times with substituents selected from the group consisting of halogen, CF₃, CN, alkoxy, alkyl, alkenyl, alkynyl, amino, nitro, and heteroaryl; or
CH═NOR′ wherein
R′ is
hydrogen or O-hydrogen;
alkyl, O-alkyl, cycloalkyl, cycloalkylalkyl, alkenyl, alkynyl or aryl, all of which may be substituted with-COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

3. The method according to claim 2, wherein R⁴ is phenyl, which is substituted once or twice with substituents selected from the group consisting of: halogen, CF₃, CN, alkoxy, cycloalkoxy, alkyl, cycloalkyl, alkenyl, alkynyl, amino, nitro, and heteroaryl.

4. The method according to claim 2, wherein R⁴ is phenyl, which is substituted once or twice with chlorine.

5. The method according to claim 1, wherein the 2,3-disubstituted tropane moiety is a compound of formula (I) 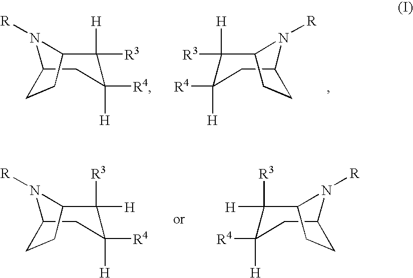
or an addition salt or N-oxide thereof, wherein
R is hydrogen, alkyl, alkenyl, alkynyl, cycloalkyl, cycloalkylalkyl, or
2-hydroxyethyl;
R³ is
CH₂—X—R′, wherein
X is O, S, or NR″, wherein
R″ is hydrogen or alkyl; and
R′ is alkyl, alkenyl, alkynyl, cycloalkyl, cycloalkylalkyl, or —CO-alkyl; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

6. The method according to claim 1, wherein the 2,3-disubstituted tropane moiety is a compound of formula (I) 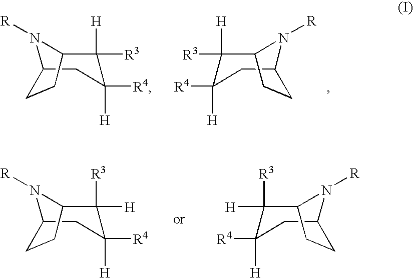
or an addition salt or N-oxide thereof, wherein
R is hydrogen, alkyl, alkenyl, alkynyl, cycloalkyl, cycloalkylalkyl, or
2-hydroxyethyl;
R³ is
CH═NOR′ wherein
R′ is
hydrogen;
alkyl, cycloalkyl, cycloalkylalkyl, alkenyl, alkynyl or aryl, all of which may be substituted with —COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

7. The method according to claim 1, wherein the 2,3-disubstituted tropane moiety is a compound of formula (I1) 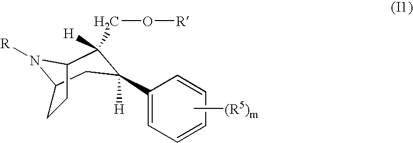
wherein
R represents a hydrogen atom or a C₁₋₆ alkyl group;
R⁵ represents a halogen atom or a CF₃ or cyano group;
R′ represents a hydrogen atom or a C₁₋₆ alkyl, or C₃₋₆-cycloalkyl-C₁₋₃-alkyl group; and
m is 0 or an integer from 1 to 3;
or a tautomer, pharmaceutically acceptable salt, solvate, or physiologically functional derivative thereof.

8. The method according to claim 7, wherein:
R represents hydrogen, or a methyl or ethyl group;
R⁵ represents fluorine, chlorine, or bromine;
R′ represents a methyl, ethyl, or n-propyl group; and
m is 1 or2.

9. The method according to claim 1, wherein the 2,3-disubstituted tropane moiety is selected from the group consisting of:
(1R,2R,3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2R,3S)-3-(3,4-Dichlorophenyl)-tropane-2-O-benzyl-aldoxime;
(1R,2R,3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2R,3S)-3-(3,4-Dichlorophenyl)-tropane-2-O-(1-ethoxycarbonyl-1,1-dimethyl-methyl)-aldoxime;
(1R,2R,3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R,3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoxime hydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)-N-Normethyl-2-methoxymethyl-3-(4-chlorophenyl)-tropane;
(1R,2R,3S)-2-Ethoxymethyl-3-(4-chlorophenyl)-tropane;
(1R,2R,3S)-N-Normethyl-2-methoxymethyl-3-(3,4-dichlorophenyl)-tropane;
(1R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2R,3S)-N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2R, 3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R,3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2R,3S)-N-Normethyl-N-allyl-2-(3-(4-pyridyl)-1,2,4-oxadiazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R,3S)-N-Normethyl-2-(3-(4-pyridyl)-1,2,4-oxadiazol-5-yl)-3-(3,4-dichlorophenyl)-tropane;
(1R,2R,3S)-N-Normethyl-N-allyl-2-(3-(3-pyridyl)-1,2,4-oxadiazol-5-yl)-3-(3,4-dichlorophenyl)-tropane;
(1R,2R,3S)-N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(4-chlorophenyl)-tropane;
(1R,2R,3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R,3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane; and
(1R,2R,3S)-2-(4-Fluoro-benzoyl)-3-(4-fluorophenyl)-tropane,
or a pharmaceutically acceptable addition salt of such 2,3-disubstituted tropane moiety.

10. The method according to claim 1, wherein the 2,3-disubstituted tropane moiety is a compound of formula (IA) or (IB) 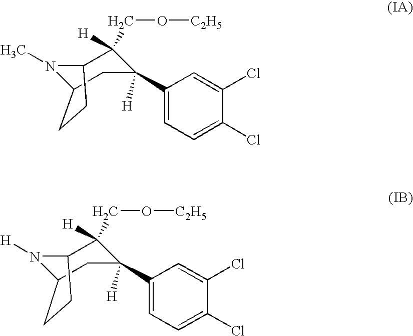
or a pharmaceutically acceptable salt thereof;

11. The method according to claim 1, wherein the 2,3-disubstituted tropane moiety is present in a weight of about 0.05 mg to about 10 mg.

12. The method according to claim 1, wherein the composition is administered orally, intravenously, intravascularly, intraperitoneally, sub-cutaneously, intramuscularly, inhalatively, topically, by patch, or by suppository.

13. The method according to claim 1, further comprising the step of administering to a human a partner compound selected from the group consisting of: D₁-, D₂-, D₃- or D₄-agonists, anorectics, lipase inhibitors, and sympathomimetics, and pharmaceutically acceptable salts, solvates, physiologically functional derivatives, combinations, and mixtures thereof.

14. The method according to claim 1, further comprising the step of administering to a human a partner compound selected from the group consisting of: adrogolide, A-86929, Rotigotine, NeurVex, nolomirole, pramipexole, talipexol, CHF 1512, (−)-stepholidine, DAR-201, diacrin/Genzyme, bromocriptine, bupropion, LEK-8829, BAM-1110, AIT-203, terguride, aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, ropinirole, SKF-82958, SKF-77434, DU 127090, SLV-308, SLV 318, NeuroCRIB, SP-1037C, spheramine, gallotrank, preclamol, DAB-452, YM-435, BP-897, ProSavin, etilevodopa, P63, A 68930, A 77636, alaptide, alentemol, Cl 1007; PD 143188, BLSI, JA 116a; JA 116, melevodopa; levodopa methyl; CHF 1301; NSC 295453; levomet, MR 708, PD 128483, RD 211, SKF 38393, SKF 81297, U 86170F, U 91356A, WAY 124486, Z 15040, silbutramine, orlistat, amfepramon-HCl, ephedrine, and combinations, and mixtures thereof.

15. A composition comprising:
a 2,3-disubstituted tropane moiety of formula IA or IB 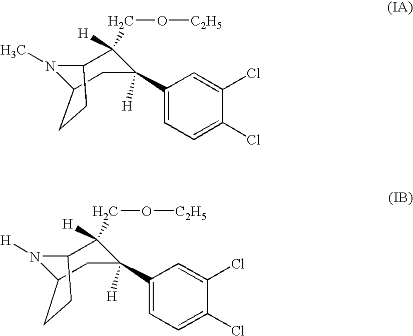
or a tautomer, pharmaceutically acceptable salt, solvate, or physiologically functional derivative thereof, that is effective for sustained reduction of body weight in a human.

16. The composition according to claim 15, wherein the 2,3-disubstituted tropane moiety is present in a weight of about 0.05 mg to about 10 mg.

17. The composition according to claim 15, wherein the 2,3-disubstituted tropane moiety is present in a weight of about 0.125 mg to about 1 mg.

18. The composition according to claim 15, wherein the composition is adapted for oral, intravenous, intravascular, intraperitoneal, sub-cutaneous, intramuscular, inhalative, topical, patch, or suppository administration.

19. The composition according to claim 15, further comprising a partner compound selected from the group consisting of: D₁-, D₂-, D₃- or D₄-agonists, anorectics, lipase inhibitors, and sympathomimetics, and pharmaceutically acceptable salts, solvates, physiologically functional derivatives, combinations, and mixtures thereof.

20. The composition according to claim 15, further comprising a partner compound selected from the group consisting of: adrogolide, A-86929, Rotigotine, NeurVex, nolomirole, pramipexole, talipexol, CHF 1512, (−)-stepholidine, DAR-201, diacrin/Genzyme, bromocriptine, bupropion, LEK-8829, BAM-1110, AIT-203, terguride, aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, ropinirole, SKF-82958, SKF-77434, DU 127090, SLV-308, SLV 318, NeuroCRIB, SP-1037C, spheramine, gallotrank, preclamol, DAB-452, YM-435, BP-897, ProSavin, etilevodopa, P63, A 68930, A 77636, alaptide, alentemol, Cl 1007; PD 143188, BLSI, JA 116a; JA 116, melevodopa; levodopa methyl; CHF 1301; NSC 295453; levomet, MR 708, PD 128483, RD 211, SKF 38393, SKF 81297, U 86170F, U 91356A, WAY 124486, Z 15040, silbutramine, orlistat, amfepramon-HCl, ephedrine, and combinations, and mixtures thereof.
